(19) United States
(12) Patent Application Publication
(10) Pub. No.: US 2018/0036446 A1
(43) Pub. Date: Feb. 8, 2018

(54) MOBILE UNIT FOR RAPID GROUP CLEANING OF UNIFORMS AND OR SPORTS GEAR AND RELATED METHOD

(71) Applicants: Adam Rice, Clairton, PA (US); Mark Rice, Jefferson Hills, PA (US)

(72) Inventors: Adam Rice, Clairton, PA (US); Mark Rice, Jefferson Hills, PA (US)

(21) Appl. No.: 15/668,581

(22) Filed: Aug. 3, 2017

Related U.S. Application Data
(60) Provisional application No. 62/370,378, filed on Aug. 3, 2016.

(51) Int. Cl. A61L 2/26; A61L 9/04; A61L 2/20; A63B 71/00; A61L 9/12

(52) U.S. Cl. CPC A61L 2/26; A63B 71/00; A61L 9/122; A61L 2/202; A61L 9/046; A61L 2202/11; A61L 2202/14; A61L 2202/15; A61L 2202/16; A61L 2209/212; A61L 2209/15

(57) ABSTRACT

A mobile unit for group exposure of equipment to an ozone gas treatment, said mobile unit including: a high capacity fan, a power generator, an ozone generator, an oxygen concentrator; an ozone level monitor; and at least one of: (i) a standing rack; and (ii) a rotating rack, each rack designed for holding a plurality of individual equipment for treatment. A method of using this mobile unit for bulk cleaning treatments is also disclosed.

### CROSS-REFERENCE TO RELATED APPLICATION

This is a perfection of U.S. Provisional Application Ser. No. 62/370,378, filed on Aug. 3, 2016, the disclosure of which is fully incorporated by reference herein.

### BACKGROUND OF THE INVENTION

Unlike the high heat/humidity “ozone action” days of pollution alerts in the city of the co-inventors, the benefits of using ozone gas, O₃, are generally known as a purifier and/or antibacterial agent. Recently, there was introduced an individualized device for cleaning one's CPAP mask, during the day.

It is also known to use ozone treatments in a one-on-one machine for cleaning sports equipment per se. See, for example, freshgear.com, sani-sport.com and/or teamcleangear.com. Some of these are provided as laundry additives and/or alternatives. Still others employ an individualized unit for “gassing” one's sports gear clean using a limited exposure to ozone gas.

It would be tedious and very time consuming to use the foregoing practices for treating an entire team's sports gear, school band uniforms and/or firehouse gear. It is further the case that prior O₃ machines required dropping off one's shoulder pads, skates, helmets, etc. for an overnight treatment in a small batch operation for pickup the next day, or days later . . . not unlike a cleaners for pressing one's shirts and suits.

What if one could devise a system for bulk cleaning uniforms and/or sports gear to reduce the germs, and smells of the same? What if such bulk cleansing took less time, or as little time as it might take to treat one's little league (or deck hockey) team to a postgame pizza or round of ice cream? And what if that cleaning “system” came TO the customer rather than lugging a dozen sets of skates and sweaty gloves to a bulk treatment facility?

This invention addresses all of the foregoing needs. It does not claim novelty in using ozone gas to cleanse (and fumigate) sports gear per se. But it DOES provide a mobile system that can deliver a rapid cleansing to a whole team's set of gear . . . dare we say: in situ?

### SUMMARY OF THE INVENTION

Rather than bringing one's STUFF to the cleaners, this invention brings the cleaner to the team . . . at the playing site, most often, or to a neighborhood, or individual residence within said neighborhood. And, rather that sitting around a treatment facility for the hours it might take to fully treat one set of pads, skates and gloves for one, maybe two players, in one of the known individualized treatment units . . . then repeating that cycle of wash-n-wait over and over until the equipment for all 12-15 players have been cleaned or “semi-sterilized” via ozone immersion, this invention enables everyone to be treated altogether. No fuss and no need to figure out ways to entertain a dozen fidgety pre-teens (or young adults) while the team's gear gets subjected to a mass/group cleansing. Nor is there a need for coaches and/or parents to prioritize whose gear gets treated in the first cycle, second . . . and all the way to the last of 10 or 12 such treatments.

This invention accomplishes the foregoing, preferred in situ treating of bulk equipment, more preferably “used” sports equipment, by driving a mobile unit or truck up to a given location for group exposures of the equipment to ozone gas (for 30 minutes or less). That truck would include: a high capacity fan, power generator, an ozone generator, an oxygen concentrator (in this instance, one housed within a steel case on a side exterior of the truck proper); an ozone level monitor; and at least one of: (i) a standing rack; and (ii) a rotating rack, each rack designed for holding a plurality of individual equipment for treatment. A method of using this truck for such bulk treatments, on location, is also disclosed.

### SUMMARY OF THE DRAWINGS

Further features, objectives and advantages of this invention will be made clearer with the following Detailed Description made with reference to the accompanying drawings in which:

FIG. 1 is a front perspective view of one mobile unit for practicing a preferred embodiment of this invention, said mobile unit, or truck, being emblazoned with the inventors' Federally registered service mark: BROZONE®;

FIG. 2 is front perspective view of a first rack employable inside the truck of FIG. 1, said rack including a plurality of loops above a plurality of legwear posts through which cleansing ozone gas is fed;

FIG. 3 is a rotating rack onto which other sports equipment may be hooked for cleansing inside the truck of FIG. 1; and

FIG. 4 is a side perspective view showing the operational panel of the FIG. 1 truck with its ozone making and monitoring units atop a generator for powering all necessary equipment for the operation of this invention.

### DETAILED DESCRIPTION OF PREFERRED EMBODIMENTS

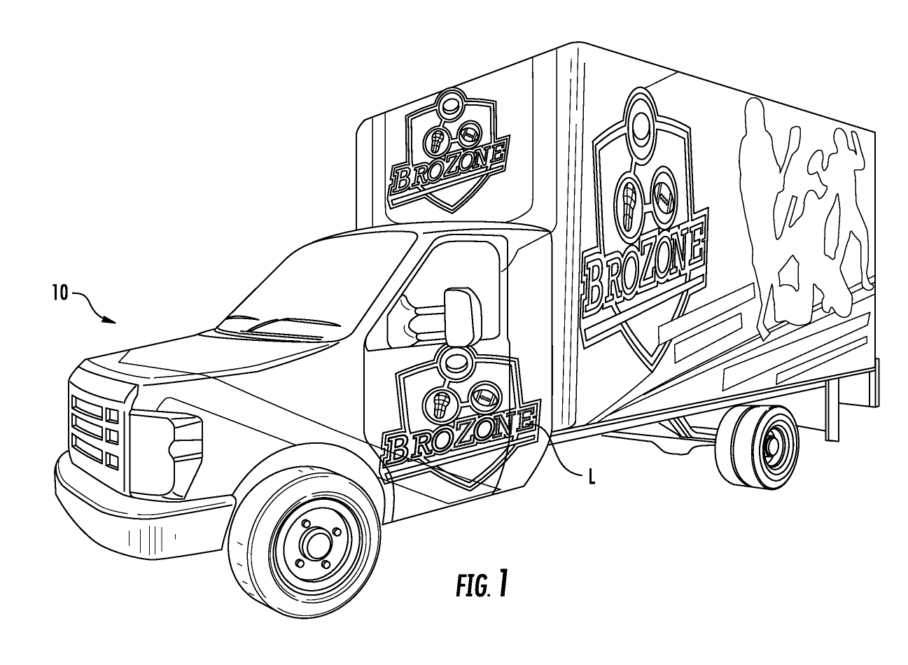

The invention employs a mobile unit or truck (like that shown as item 10 in accompanying FIG. 1). Note, the proposed franchise-able name for such a unit includes the logo L, BROZONE®, now a Federally registered service mark. On or in this truck 10, there would be situated: a high speed, high capacity fan/exhaust port F; and an ozone generator 20 such as one that can produce between: (a) about 4 liters/min (LPM) at a flow rate of about 3% by weight; to (b) about 4 grams/hour from a 5 liters/min LPM at a flow rate of about 0.79% by weight. A representative model is made and sold by Oxidation Technologies L.L.C. as part of its OXG Series of ozone generators.

In addition, each mobile unit would include an oxygen concentrator 30 that would be capable of producing about 15 SCFH (sq. cu. ft./hr.) from ambient air—for producing a nominally 93% pure oxygen intermediate for feeding into the aforementioned ozone generator 20.

It is believed that a typical treatment “cycle” should include ozone exposure for at least 10 minutes at a saturation level of at least 5 PPM to be bactericidal. Now, in an enclosed truck, it may take some time to ramp up to that exposure level and some time to ramp down therefrom. A typical, total treatment time of about 20 minutes should suffice. Upon evacuating the ozone from the mobile unit, via an exhaust fan, the franchise operator may safely re-enter his/her truck and dispense with all of the treated clothes and/or equipment (sports/safety, etc.).

Note, that this is a fully self-sufficient system. It DOES include its own power generator P. But, it does not require any special (internal or external) source of ozone supply, from tanks or the like. Nor does it generate any residue/end product that would require special post operation or shift treatment, handling and/or disposal. The ozone that would get exhausted from fan F of truck 10 can be safely released into the environment as it will revert to breathable oxygen without any special processing.

Ideally, each unit will need an ozone monitor 40 that will serve two main purposes: First, monitoring the actual amount of ozone levels IN the treatment area (i.e., the truck interior) so that operators know that they are providing clinically relevant concentrations of ozone every time. Secondly, it will alert the operators, via alarm 50, when the previously-evacuated truck 10 is safe to reenter after a treatment cycle has been completed. One such monitor is the OS-6 Model made and sold by ECO Sensors. To the knowledge of one component supplier, Applicants are the only company actually using sensors to monitor and maintain a study-recommended level of ozone over a clinically recommended time period for ensuring beneficial bactericidal effects.

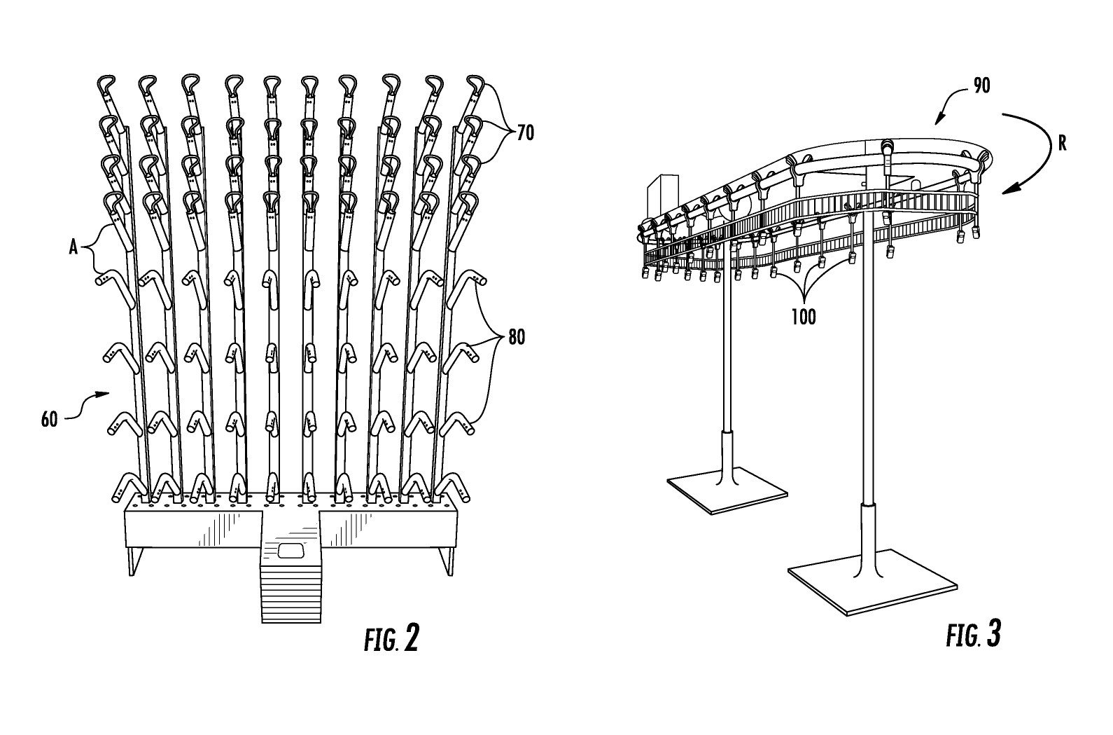

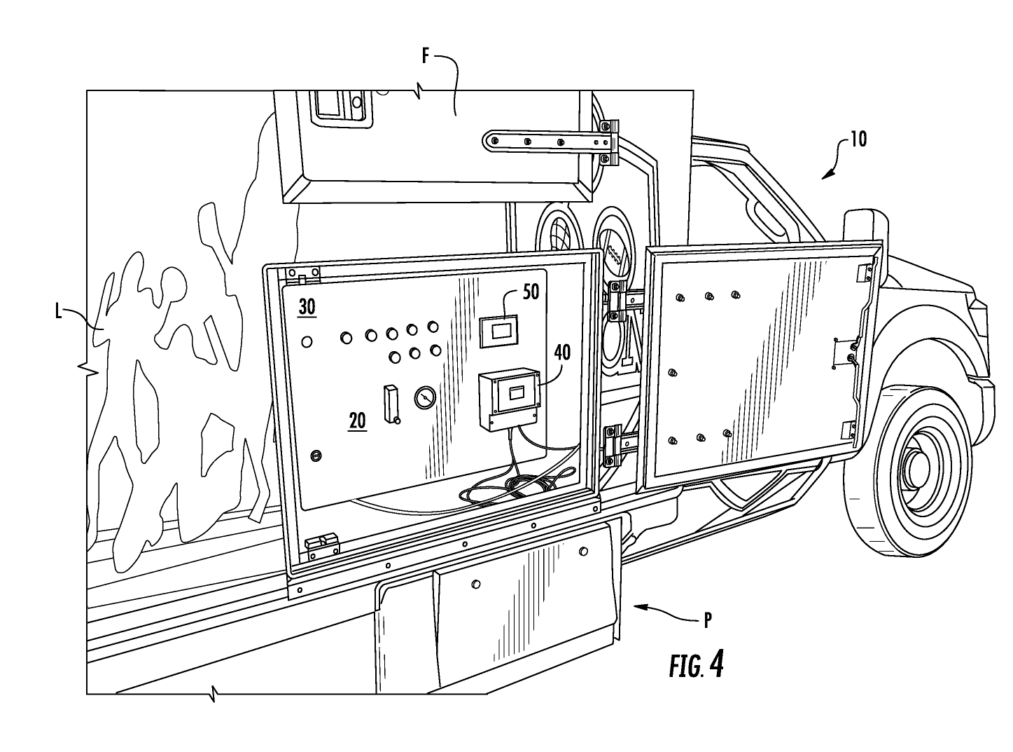

For enabling the bulk cleaning of a whole team's gear (skates, gloves, etc.), band uniforms and/or a whole firehouse full of coats, pants, etc., the BROZONE® truck would have a two-fold means for mass ozone treating of sports equipment. A skate and glove rack like that depicted as item 60 in accompanying FIG. 2, with its plurality of loops 70 and posts 80, is meant to hold up to 20 pairs/sets of player gloves and skates for a common ozone cleansing, or cleaning treatment. Additional skate/shoe cleansing and/or equipment (gloves, etc.) may be accomplished if the aforementioned loops 70 and/or posts 80 would include a plurality of apertures A that can serve as individualized ozone dispenser pathways into (and then back out of) the respective items being treated. Elsewhere, within truck 10, there would be situated a rack like item 90 in FIG. 3. That rack 90 can be timed to rotate in one (or more) directions, clockwise (and/or counterclockwise), as indicated by arrow R, about the interior of truck 10. Rack 90 would have a plurality of hooks 100 onto which could be temporarily clamped individual shoulder pads and/or uniform tops/shirts for similar (albeit rotational) bulk ozone exposure.

Typical sports include hockey, lacrosse, football and baseball/softball. Then again, the shoes of most any sport can also be cleaned/sanitized hereby. This includes wrestling, track, tennis, basketball and volleyball. The same invention could also be used for still other groups sets of uniform/equipment including but not limited to: band uniforms and/or those of the police, military and/or various municipality fire departments. It could also be used to treat musical instruments to a limited extent though it is recommended that such instruments be physically scrubbed before being subjected to the ozone treatments prescribed above. Lastly, it should be noted that, as a truly mobile cleaning unit, this same vehicle could be driven to a given street or personal residence for making “house call” cleanings of one's (or one family's) collection of sports equipment and activewear (i.e., uniforms, etc.). It is the very motto of our organization that “If You Wear It and It Stinks, We Can Sanitize It and Deodorize It” through our unique mobile processing unit described above.

Preliminarily, it is expected that a typical beginning-to-end treatment cycle for a whole load (the entire team's) of equipment (sports and/or safety) may take less than 30 minutes, or about a third of hour at a “typical” rate of ozone gas circulation within the parked mobile unit/truck. It is preferred to keep ozone gas treatments at a rate above about 5 ppm, for at least about 10 minutes before cool downs and operator redistribution of “clean” sports gear BACK to the individual team players waiting nearby.

Having described the presently preferred embodiments, it is to be understood that the scope of this invention may be otherwise protected by the subject matter of the following system and method claims.

What is claimed is:

1. A mobile system for group cleaning sports equipment for a plurality of players, said system comprising:
(a) a truck having a high capacity fan and a power generator;
(b) an ozone generator and an oxygen concentrator;
(c) an ozone level monitor; and
(d) at least one of: (i) a standing rack; and (ii) a rotating rack, each rack designed for holding a plurality of individual sports equipment for treatment.

2. The mobile system of claim 1, which further includes an ozone level alarm.

3. The mobile system of claim 1 wherein the standing rack includes a plurality of loops and posts, each of which may hold a sports glove, skate or shoe for treatment.

4. The mobile system of claim 3 wherein said loops and posts include apertures through which ozone gas may circulate.

5. The mobile system of claim 1 wherein the rotating rack includes a plurality of clips or hooks for holding shoulder pads or uniforms during treatment.

6. The mobile system of claim 1, which is adapted to treat a load of equipment with ozone exposure for less than 30 minutes.

7. The mobile system of claim 6, which is adapted to treat the load of equipment for about 10 minutes at an exposure level of about 5 PPM or more.

8. The mobile system of claim 1, which may be driven to a residence or to a hockey rink, sports field, track or gymnasium for group cleaning a team's equipment at that location.

9. The mobile system of claim 1, which may be used to treat uniforms for a school band, police, municipal fire department and military units.

10. A method for in situ bulk treating multiple sets of equipment for odor and bacterial exposure, said method comprising:
(a) providing a mobile truck that includes a high capacity fan, a power generator, an ozone generator, an oxygen concentrator; an ozone level monitor; and at least one of: (i) a standing rack; and (ii) a rotating rack, each rack designed for holding a plurality of equipment for treatment;
(b) delivering the mobile truck to a location where the equipment is being used or stored;
(c) situating the equipment of six or more individuals onto one or more racks within the mobile truck;
(d) exposing the equipment to ozone gas at concentrations of about 5 PPM or more for at least about 10 minutes;
(e) allowing the ozone gas to exit the mobile truck after treatment of the equipment; and
(f) returning the equipment, after treatment, to the individuals.

11. The method of claim 10 wherein the location is selected from the group consisting of: a private residence, a neighborhood block, a hockey facility, football/soccer or lacrosse field, a baseball diamond, an outdoor track and a gymnasium.

12. The method of claim 10 wherein the location is a firehouse, police station or military base.

13. The method of claim 10 wherein the location is a school for treating band uniforms.

14. The method of claim 10 wherein the location is a school for treating band instruments.

15. A method for in situ bulk treating multiple sets of sports equipment for odor and bacterial exposure, said method comprising:
(a) providing a mobile truck that includes a high capacity fan, a power generator, an ozone generator, an oxygen concentrator; an ozone level monitor; and at least one of: (i) a standing rack; and (ii) a rotating rack, each rack designed for holding a plurality of sports equipment for treatment;
(b) delivering the mobile truck to a location where the sports equipment is being used or stored;
(c) situating the sports equipment of six or more individuals onto one or more racks within the mobile truck;
(d) exposing the sports equipment to ozone gas at concentrations of about 5 PPM or more for at least about 10 minutes;
(e) allowing the ozone gas to exit the mobile truck after treatment of the sports equipment; and
(1) returning the sports equipment, after treatment, to the individuals.

16. The method of claim 15 wherein the location is selected from the group consisting of: a residence, a hockey facility, football/soccer or lacrosse field, a baseball diamond, an outdoor track and a gymnasium.
